(19) United States
(12) Patent Application Publication
(10) Pub. No.: US 2023/0341391 A1
(43) Pub. Date: Oct. 26, 2023

(54) SARS-COV-2 (COVID-19) ANTIBODY TEST ON SALIVA AND BLOOD USING EFIRM TECHNOLOGY

(71) Applicant: THE REGENTS OF THE UNIVERSITY OF CALIFORNIA, Oakland, CA (US)

(72) Inventors: David Wong, Beverly Hills, CA (US); Charles Strom, San Clemente, CA (US)

(21) Appl. No.: 17/999,417

(22) PCT Filed: May 21, 2021
(86) PCT No.: PCT/US2021/033601

§ 371 Date: Nov. 21, 2022

Related U.S. Application Data
(60) Provisional application No. 63/028,313, filed on May 21, 2020.

(51) Int. Cl. G01N 33/569; G01N 33/543; G01N 27/327

(52) U.S. Cl. CPC G01N 33/56983; G01N 33/5438; G01N 27/3276; G01N 27/3271; G01N 2469/20; G01N 2333/165

(57) ABSTRACT

A liquid biopsy system and method for the detection of SARS-CoV-2 antibodies in bodily fluids is described. In particular, the system is suitable for detecting SARS-CoV-2 antibodies in a saliva sample of a subject.

### CROSS REFERENCE TO RELATED APPLICATIONS

This application claims priority to U.S. Provisional Application No. 63/028,313, filed May 21, 2020, which is hereby incorporated by reference herein in its entirety.

### BACKGROUND OF THE INVENTION

The global pandemic of SARS-COV-2 infection and its resultant disease, COVID-19, has had a global disruptive effect and has stressed health care systems. It has become clear that many patients infected with the SARS-COV-2 virus have mild symptoms or are asymptomatic. Therefore, it is possible that there is a background herd immunity in any population. As governments weigh the loosening of social distancing policies, it is important to know the background level of immunity in the population in order to inform predictive modeling algorithms. In addition, health care providers, first responders, and other essential workers may have already been infected and have immunity to the virus. Therefore, it is important to have means of noninvasive large scale testing for SARS-COV-2 antibodies. All currently available tests involve blood sampling. Unfortunately attempts to validate a home finger stick method have failed and patient acceptance of self finger sticking has been historically poor.

Thus, there is a need in the art for diagnostic systems and methods that are non-invasive, always available, include minimal or no sample preparation, and provide immediate information on infection and immunity status. The present invention satisfies this need.

### SUMMARY OF THE INVENTION

In one embodiment, the invention relates to a system for detecting a SARS-CoV-2 antibody in a sample, comprising: a) a multi-well plate comprising an array of sensors, wherein each well comprises an electrode chip including a working electrode, a counter electrode, and a reference electrode; wherein the working electrode of at least one unit is coated with a conducting polymer; b) at least one SARS-CoV-2 capture antigen, wherein the at least one capture antigen is embedded or functionalized in the conducting polymer; c) at least one labeled detector molecule, and further wherein the detector molecule is biotin labeled; d) a multi-well plate washer; and e) a multi-channel electrochemical reader which controls an electrical field applied onto the array sensors and reports the amperometric current simultaneously.

In one embodiment, at least one capture antigen is a SARS-CoV-2 spike 1 antigen, a SARS-CoV-2 spike 2 antigen, a SARS-CoV-2 envelope antigen, nucleocapsid protein, a protein synthesized from a SARS-CoV-2 open reading frame (ORF), a fragment thereof or any combination thereof. In one embodiment, the capture antigen comprises a combination of SARS-CoV-2 spike 1 antigen and SARS-CoV-2 spike 2 antigen.

In one embodiment, at least one detector molecule is a secondary antibody specific for binding to an antibody constant region. In one embodiment, the system comprises a combination of at least two detector molecules wherein the at least two detector molecules are secondary antibodies specific for binding to an antibody constant region. In one embodiment, the system comprises a combination of at least two detector molecules wherein the at least two detector molecules are secondary antibodies specific for binding to an IgG and an IgA constant region.

In one embodiment, the invention relates to a method of detecting a SARS-CoV-2 antibody in a subject comprising: obtaining at least one sample of the subject; mixing a first portion of the at least one sample with a solution comprising a labeled detector molecule; adding the mixture to a single well of a multi-well plate for use in a system for detecting a SARS-CoV-2 antibody in a sample, comprising: a) a multi-well plate comprising an array of sensors, wherein each well comprises an electrode chip including a working electrode, a counter electrode, and a reference electrode; wherein the working electrode of at least one unit is coated with a conducting polymer; b) at least one SARS-CoV-2 capture antigen, wherein the at least one capture antigen is embedded or functionalized in the conducting polymer; c) at least one labeled detector molecule, and further wherein the detector molecule is biotin labeled; d) a multi-well plate washer; and e) a multi-channel electrochemical reader which controls an electrical field applied onto the array sensors and reports the amperometric current simultaneously, wherein each well of the multi-well plate comprises an electrode chip comprising a working electrode, a counter electrode, and a reference electrode; wherein the working electrode is coated with a conducting polymer embedded with a capture antigen; applying a cyclic square-wave electric field to the electrode chip; and measuring the current in the electrode chip, wherein a change in current is correlated to the presence of a SARS-CoV-2 antibody in the sample.

In one embodiment, the method further comprises at least one washing step, wherein the multi-well plate is washed using an automated plate washer.

In one embodiment, the method further comprises amplifying the signal, method comprising the steps of: a) mixing a first portion of the at least one sample with a solution comprising a biotin labeled detector molecule; b) adding the mixture to a single well of a multi-well plate for use in a system for detecting a SARS-CoV-2 antibody in a sample, comprising: i) a multi-well plate comprising an array of sensors, wherein each well comprises an electrode chip including a working electrode, a counter electrode, and a reference electrode; wherein the working electrode of at least one unit is coated with a conducting polymer; ii) at least one SARS-CoV-2 capture antigen, wherein the at least one capture antigen is embedded or functionalized in the conducting polymer; iii) at least one labeled detector molecule, and further wherein the detector molecule is biotin labeled; iv) a multi-well plate washer; and v) a multi-channel electrochemical reader which controls an electrical field applied onto the array sensors and reports the amperometric current simultaneously, wherein each well of the multi-well plate comprises an electrode chip comprising a working electrode, a counter electrode, and a reference electrode; wherein the working electrode is coated with a conducting polymer embedded with a capture antigen; c) applying a cyclic square-wave electric field to the electrode chip; d) adding a first round of streptavidin bound horseradish peroxidase (HRP) to the well, e) adding a biotin labeled anti-HRP antibody to the well, f) adding a second round of streptavidin bound HRP to the well, and g) measuring the current in the electrode chip, wherein a change in current is correlated to the presence of a SARS-CoV-2 antibody in the sample.

In one embodiment, at least one capture antigen is a SARS-CoV-2 spike 1 antigen, a SARS-CoV-2 spike 2 antigen, a SARS-CoV-2 envelope antigen, nucleocapsid protein, any protein synthesized from a SARS-CoV-2 open reading frame (ORF), a fragment thereof or any combination thereof. In one embodiment, the capture antigen comprises a combination of SARS-CoV-2 spike 1 antigen and SARS-CoV-2 spike 2 antigen.

In one embodiment, at least one detector molecule is a secondary antibody specific for binding to an antibody constant region. In one embodiment, the method comprises contacting the sample with a combination of at least two detector molecules wherein the at least two detector molecules are secondary antibodies specific for binding to an antibody constant region. In one embodiment, the method comprises contacting the sample with a combination of a combination of at least two detector molecules wherein the at least two detector molecules are secondary antibodies specific for binding to an IgG and an IgA constant region.

In one embodiment, the sample is a saliva sample, a blood sample, a plasma sample or a serum sample.

In one embodiment, the method further comprises diagnosing a subject as having, being at risk of spreading, having been exposed to or having immunity to SARS-CoV-2 infection or COVID-19 when a SARS-CoV-2 antibody is detected in the sample from the subject. In one embodiment, the method further comprises administering a therapeutic treatment for COVID-19 to the subject when the SARS-CoV-2 antibody is detected.

In one embodiment, the method further comprises diagnosing a subject as being at risk of SARS-CoV-2 infection or COVID-19 when a SARS-CoV-2 antibody is not detected in the sample from the subject. In one embodiment, the method further comprises administering a prophylactic treatment for COVID-19 to the subject when the SARS-CoV-2 antibody is not detected.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a schematic diagram of the EFIRM assay system for detection of SARS-COV-2 antibodies.

FIG. 2 depicts linearity experiments for the detection of recombinant human anti-S1 antibody using the EFIRM assay with the S1 concentrations varying between 160 ng/ml and 600 ng/ml. capture antigen or the combination of S1 and S2 capture antigens.

FIG. 3 depicts linearity experiments for the detection of recombinant human anti-S1 antibody using the EFIRM assay with the S1 concentrations varying between 25 ng/ml and 300 ng/ml.

FIG. 4: EFIRM Saliva test for COVID-19 IgG plus IgA antibodies using saliva samples obtained from 3 patients with documented COVID-19 infections between 3-6 weeks prior to testing.

FIG. 5: Competition Experiment on patient saliva samples demonstrating that addition of exogenous S1 antigen at varying concentrations diminishes the EFIRM signal in a dose dependent manner.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical biomarker detection systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein the terms “alteration,” “defect,” “variation,” or “mutation,” refers to a mutation in a gene in a cell that affects the function, activity, expression (transcription or translation) or conformation of the polypeptide that it encodes. Mutations encompassed by the present invention can be any mutation of a gene in a cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing the mRNA (splice site mutation) or cause a shift in the reading frame (frameshift).

The term “amplification” refers to the operation by which the number of copies of a target nucleotide sequence present in a sample is multiplied.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The level of a SARS-CoV-2 antibody “significantly” differs from the level of the SARS-CoV-2 antibody in a reference sample if the level of the SARS-CoV-2 antibody in a sample from the patient differs from the level in a sample from the reference subject by an amount greater than the standard error of the assay employed to assess the SARS-CoV-2 antibody, for example, by at least 5%, 10%, 25%, 50%, 75%, or 100%.

The term “control or reference standard” describes a material comprising one, or a normal, low, or high level of one of more SARS-CoV-2 antibody, such that the control or reference standard may serve as a comparator against which a sample can be compared.

A “disease” is a state of health of an animal wherein the animal cannot maintain homeostasis, and wherein if the disease is not ameliorated then the animal's health continues to deteriorate.

As used herein, an “instructional material” includes a publication, a recording, a diagram, or any other medium of expression which can be used to communicate the usefulness of a component of the invention in a kit for detecting a SARS-CoV-2 antibody disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The term “label” when used herein refers to a detectable compound or composition that is conjugated directly or indirectly to a molecule to generate a “labeled” molecule. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The “level” of one or more antibody means the absolute or relative amount or concentration of the antibody in the sample.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “providing a prognosis” refers to providing a prediction of the probable course and outcome of COVID-19, including, e.g., prediction of immunity to reinfection. The methods are used to devise a suitable therapeutic plan, e.g., by indicating whether or not the subject would benefit from vaccination or another treatment regimen.

A “reference level” of a antibody means a level of the antibody that is indicative of a particular disease state, phenotype, or lack thereof, as well as combinations of disease states, phenotypes, or lack thereof. A “positive” reference level of an antibody means a level that is indicative of a particular disease state or phenotype. A “negative” reference level of an antibody means a level that is indicative of a lack of a particular disease state or phenotype.

“Sample” or “biological sample” as used herein means a biological material isolated from an individual. The biological sample may contain any biological material suitable for detecting the desired antibody, and may comprise cellular and/or non-cellular material obtained from the individual.

“Standard control value” as used herein refers to a predetermined amount of a particular protein or nucleic acid that is detectable in a sample, such as a saliva sample, either in whole saliva or in saliva supernatant. The standard control value is suitable for the use of a method of the present invention, in order for comparing the amount of a protein or nucleic acid of interest that is present in a saliva sample. An established sample serving as a standard control provides an average amount of the protein or nucleic acid of interest in the saliva that is typical for an average, healthy person of reasonably matched background, e.g., gender, age, ethnicity, and medical history. A standard control value may vary depending on the protein or nucleic acid of interest and the nature of the sample (e.g., whole saliva or supernatant).

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### Description

This invention is a method of measuring levels of human antibodies present in bio-fluids including, but not limited to, saliva and blood, to antigens from infectious agents. Infectious agents include, but are not limited to, viruses, bacteria, parasites, protozoa and fungi. In one embodiment, the infectious agent is SARS-CoV-2 virus, the causative agent in COVID-19 infection. The method uses the method known as Electric Field Induced Release and Measurement (EFIRM). This invention may be used for the purposes of epidemiologic investigation or to determine the immunity status of symptomatic or asymptomatic individuals.

In one embodiment, the invention relates to a rapid and accurate polymer-based electrochemical platform array for detection of a SARS-CoV-2 antibody from at least one biological sample, such as a saliva sample or blood sample, that are indicative of an infection with or immunity to a disease or disorder associated with SARS-CoV-2, for example, COVID-19. While the present invention is described generally for the testing of a saliva sample or blood sample, it should be appreciated that any biological fluid sample may be used, or even other tissue types, provided such alternative sample types carry the antibodies to be detected. In some embodiments, the antibody is specific for a SARS-CoV-2 antigen including but not limited to, SARS-CoV-2 envelope antigen, SARS-CoV-2 virus spike protein 1, SARS-CoV-2 virus spike proteins 2, a combination of SARS-CoV-2 virus spike proteins 1 and 2, nucleocapsid protein, or any proteins synthesized from the open reading frames (ORF), or any other SARS-CoV-2 virus antigen.

The noninvasive detection of SARS-CoV-2 antibodies in a subject via the present invention enables clinicians to identify the presence of SARS-CoV-2 infection or immunity in a fast, economical and non-invasive manner.

As contemplated herein, the present invention includes a multiplexing electrochemical sensor for detecting antibodies in multiple samples simultaneously. The device utilizes a small sample volume with high accuracy. In addition, multiple capture antigens can be combined on each electrochemical sensor to detect antibodies to multiple antigens simultaneously on the device with single sample loading. The device may significantly reduce the cost to the health care system.

In one embodiment, the electrochemical sensor is an array of electrode chips (EZ Life Bio, USA). In one embodiment, each unit of the array has a working electrode, a counter electrode, and a reference electrode. The three electrodes may be constructed of bare gold or other conductive material before the reaction, such that the specimens may be immobilized on the working electrode. Electrochemical current can be measured between the working electrode and counter electrode under the potential between the working electrode and the reference electrode. The potential profile can be a constant value, a linear sweep, or a cyclic square wave, for example. An array of plastic wells may be used to separate each three-electrode set, which helps avoid the cross contamination between different sensors. In one embodiment, a three-electrode set is in each well of a 96 well gold electrode plate. A conducting polymer may also be deposited on the working electrodes as a supporting film, and in some embodiments, as a surface to functionalize the working electrode. As contemplated herein, any conductive polymer may be used, such as polypyrroles, polanilines, polyacetylenes, polyphenylenevinylenes, polythiophenes and the like.

In one embodiment, a cyclic square wave electric field is generated across the electrode within the sample well. In certain embodiments, the square wave electric field is generated to aid in polymerization of one or more capture antigens to the polymer of the sensor. In certain embodiments, the square wave electric field is generated to aid in the hybridization of the capture antigens with the target molecule to be detected and/or detector molecule. The positive potential in the csw E-field helps the molecules accumulate onto the working electrode, while the negative potential removes the weak nonspecific binding, to generate enhanced specificity. Further, the flapping between positive and negative potential across the cyclic square wave also provides superior mixing during incubation, without disruption of the desired specific binding, which accelerates the binding process and results in a faster test or assay time. In one embodiment, a square wave cycle may consist of a longer low voltage period and a shorter high voltage period, to enhance binding partner hybridization within the sample. While there is no limitation to the actual time periods selected, examples include 0.15 to 60 second low voltage periods and 0.1 to 60 second high voltage periods. In one embodiment, each square-wave cycle consists of 1 s at low voltage and 1 s at high voltage. For hybridization, the low voltage may be around −200 mV and the high voltage may be around +500 mV. In some embodiments, the total number of square wave cycles may be between 2-50. In one embodiment, 5 cyclic square-waves are applied for each surface reaction. With the csw E-field, both the polymerization and hybridization are finished on the same chip within minutes. In some embodiments, the total detection time from sample loading is less than 30 minutes. In other embodiments, the total detection time from sample loading is less than 20 minutes. In other embodiments, the total detection time from sample loading is less than 10 minutes. In other embodiments, the total detection time from sample loading is less than 5 minutes. In other embodiments, the total detection time from sample loading is less than 2 minutes. In other embodiments, the total detection time from sample loading is less than 1 minute.

A multi-channel electrochemical reader (EZ Life Bio) controls the electrical field applied onto the array sensors and reports the amperometric current simultaneously. In practice, solutions can be loaded onto the entire area of the three-electrode region including the working, counter, and reference electrodes, which are confined and separated by the array of plastic wells. After each step, the electrochemical sensors can be rinsed with ultrapure water or other washing solution and then dried, such as under pure N2. In some embodiments, the sensors are single use, disposable sensors. In other embodiment, the sensors are reusable.

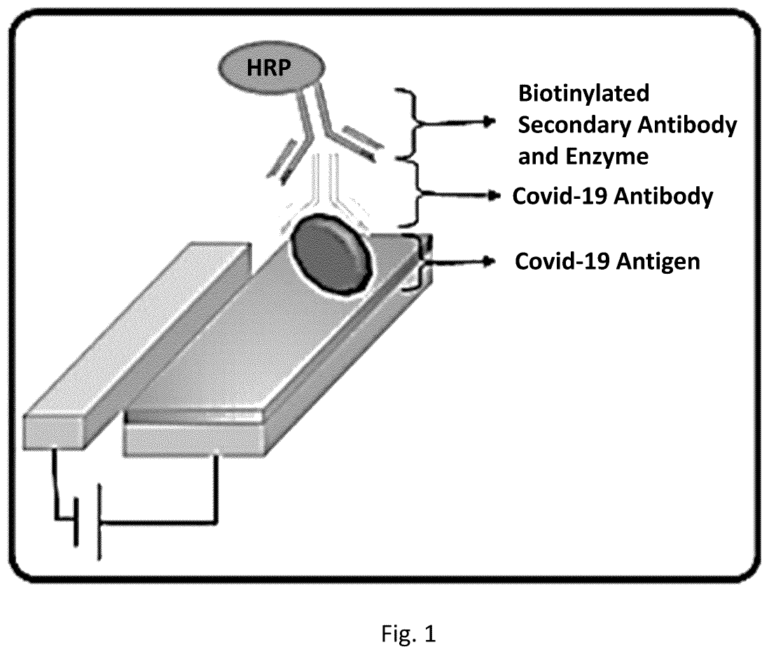

In one embodiment, the present invention is based on the affinity between a capture antigen, a target antibody and a detector molecule, as shown in FIG. 1. As contemplated herein, the assay platform may be organized as any type of affinity binding assay or immunoassay, as would be understood by those skilled in the art.

In one embodiment, at least one capture antigen is immobilized in a conductive polymer gel in the bottom of the 96 well gold electrode plate. Capture antigens embedded in the conductive polymer or otherwise used to functionalize the working electrode surface, and detector molecules mixed with the sample may be constructed according to any protocol known in the art for the generation of probes.

The capture antigen or detector molecule of the system may be any one of a nucleic acid, protein, small molecule, and the like, which specifically binds one or more antibody against an antigen from an infectious agent.

In one embodiment, the capture antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

Bacterial Antigens

The capture antigen can be a bacterial antigen or fragment or variant thereof. The bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram positive bacterium or a gram negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, an halophile, or an osmophile.

The bacterium can be an anthrax bacterium, an antibiotic resistant bacterium, a disease causing bacterium, a food poisoning bacterium, an infectious bacterium, Salmonella bacterium, Staphylococcus bacterium, Streptococcus bacterium, or Tetanus bacterium. The bacterium can be a mycobacteria, Clostridium tetani, Yersinia pestis, Bacillus anthracis, methicillin-resistant Staphylococcus aureus (MRSA), or Clostridium difficile.

Viral Antigens

The capture antigen can be a viral antigen, or fragment thereof, or variant thereof. The viral antigen can be from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from human immunodeficiency virus (HIV), Chikungunya virus (CHIKV), dengue fever virus, papilloma viruses, for example, human papillomoa virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, Severe acute respiratory syndrome-related coronavirus (SARS), Middle East respiratory syndrome-related coronavirus (MERS), Severe acute respiratory syndrome-related coronavirus 2 (SARS CoV 2) or a cancer causing virus.

Parasitic Antigens

The capture antigen can be a parasite antigen or fragment or variant thereof. The parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing any one of the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

Fungal Antigens

The capture antigen can be a fungal antigen or fragment or variant thereof. The fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

SARS-CoV-2 Capture Antigen

In one embodiment, the capture antigen is a SARS-CoV-2 antigen, or fragment thereof. In one embodiment, the capture antigen is a SARS-CoV-2 envelope antigen, SARS-CoV-2 virus spike protein 1, SARS-CoV-2 virus spike proteins 2, a combination of SARS-CoV-2 virus spike proteins 1 and 2, nucleocapsid protein, or any proteins synthesized from the open reading frames (ORF), or any other SARS-CoV-2 virus antigen, a fragment thereof or any combination thereof.

Detector Molecule

In one embodiment, the detector molecule is an antibody specific for binding to a constant region of a target antibody (i.e., a secondary antibody). In one embodiment, the detector molecule is an antibody specific for binding to an IgG, IgA, IgE, IgD or IgM constant region of an antibody. In one embodiment, the antibody is a human antibody. In one embodiment, the system uses a combination of two or more detector molecules, wherein the two or more detector molecules are antibodies specific for binding to an IgG, IgA, IgE, IgD or IgM constant region of an antibody. In one embodiment, two or more detector molecules are specific for a combination of IgG, IgA, IgE, IgD or IgM constant regions of a human antibody. In one embodiment, two or more detector molecules are specific for a combination of IgG and IgA constant regions.

It should be appreciated that any number of capture probes specific for binding to one or more additional biomarkers can be integrated to the assay platform, including, without limitation, 1, 2, 4, 8, 16, 32 or 64 biomarkers per array. The one or more additional biomarker may be any one of a nucleic acid, protein, small molecule, antibody, antibody fragment and the like which are of interest and are present in the sample. In correlation, the one or more additional capture probes may be any one of a nucleic acid, protein, small molecule, antibody, antibody fragment and the like, which specifically binds one or more markers of interest. In one embodiment, one or more capture probes are oligonucleotides or polynucleotides comprising a region that is substantially complementary to a nucleic acid marker of an infectious agent. For example, in a particular embodiment, one or more capture probes are oligonucleotides or polynucleotides comprising a region that is substantially complementary to a nucleic acid marker of the SARS-CoV-2 virus. Methods for designing and formulating oligonucleotide probes are well-known in the art. In one embodiment, one or more capture probes are antibodies or antibody fragments that specifically bind to a protein marker of SARS-CoV-2 infection, such as a SARS-CoV-2 protein (e.g., S1, S2 or envelope protein).

In one embodiment, one or more additional detector molecules are included in the system specific for binding and detection of one or more additional biomarker. The one or more additional detector molecules may be any one of a nucleic acid, protein, antibody, antibody fragment, small molecule, and the like, which binds to one or more markers of interest. The detector molecules can be labeled, such as with fluorescein isothiocyanate, or any other label known in the art. In one embodiment, the detector molecules contain a biotinylated nucleotide to allow streptavidin binding. The capture antigen is first copolymerized onto the bare gold electrode by applying a cyclic square wave electric field. For example, for each cycle during copolymerization, the electric field can be set to +350 mV for 1 s and +950 mV for 1 s. In total, polymerization may proceed for 5 cycles of 10 s, or however long is deemed necessary.

After polymerization, the sensor chip can be rinsed and dried for subsequent sample measurement. Samples, such as a cell-culture medium, a blood sample or a saliva sample, can be mixed with the detector molecules and transferred onto the electrodes. Hybridization is then carried out at low and high voltage cycles, such as −200 mV for 1 s and +500 mV for 1 s. The total hybridization time can be 5 cycles for 10 s, for example. Next, the label is detected based on the label type. For example, an anti-fluorescein antibody conjugated to horseradish peroxidase in casein-phosphate-buffered saline can be used, and the 3,3′,5,5′-tetramethylbenzidine substrate for horseradish peroxidase can be loaded, and the amperometric signal measured.

In one embodiment, the detector molecule comprises a detectable label which induces a change in current of the sensor, thereby indicating the hybridization of the detector molecule, and associated SARS-CoV-2 antibody, with the capture antigen. In certain embodiments, the detectable label itself may be sufficient to alter the current of the sensor. In certain embodiments, the detectable label induces the change in current when it comes into contact with an exogenous reactant. For example, the detectable label may react with the reactant to produce a local change sensed by the electrodes of the sensor to produce an amperometric signal. Therefore, in certain embodiments, the reactant is added to the sensor prior, during, or after the application of the sample to the sensor.

In certain embodiments, the detectable label is directly conjugated to the detector molecule. In another embodiment, the detectable label is bound to the detector molecule via an intermediate tag or label of the probe. In some embodiments, the detectable label is a modified nucleotide containing biotin incorporated into the detector molecule during synthesis. For example, in one embodiment, the detector molecule comprises a tag, label, or epitope, which can be used to bind to an antibody or other binding compound harboring the detectable label described above.

Examples of detectable labels and reactants to produce a local change in an electrochemical sensor are well known in the art. In one embodiment, the detectable label comprises HRP and the reactant is TMB, which react to generate an amperometric signal. In another embodiment, the detectable label comprises urease, while the reactant comprises urea.

In one embodiment, the signal is amplified using multiple rounds of HRP. In on embodiment, 1) a biotin labeled detector molecule is contacted with a first round of HRP in the form of streptavidin bound HRP, 2) the complexed HRP molecule is contacted with a biotin labeled Anti-HRP antibody, and 3) a second round of streptavidin bound HRP is added to amplify the signal. In one exemplary embodiment, the detector molecules are mixed with casein-phosphate-buffered saline at a 1:100 dilution and transferred onto the electrodes. Hybridization is performed at 300 mV for 1 second and 500 mV for 1 second for 150 cycles at room temperature. Subsequently, streptavidin Poly-HRP is mixed with casein-phosphate-buffered saline at a 1:1000 ratio and incubated on the electrodes for 30 minutes at room temperature. After the addition of HRP, Anti-HRP antibody with casein-phosphate-buffered saline is added, followed by a 30 minute incubation at room temperature and a wash-off with PBS-T buffer. Subsequently, Streptavidin Poly-HRP80 Conjugate mixed with casein-phosphate-buffered saline is added and incubated for 30 minutes to increase the amount of available HRP molecules. This method, results in increased signal amplification, allowing for increased sensitivity and specificity of the eLB system. In some embodiments, one or more washing steps are performed. In some embodiments, the plate is washed in an automated 96 well plate washer, in which the existing liquid is aspirated from each well of the microtiter plate, and then a wash butter dispensed into each well. In one embodiment, the wash buffer is then aspirated and this is repeated for at least one additional cycle.

Due to the enhanced sensitivity of the present invention, very small volumes may be used to perform the desired assays. For example, the biological sample size from the subject may be between 5-100 microliters. In one embodiment, the sample size need only be about 40 microliters. There is no limitation to the actual or final sample size to be tested.

The present invention also relates to a method of detecting one or more antibodies or antigens associated with, or indicative of, an infectious agent or a disease or disorder associated with an infectious agent. Exemplary infectious agents include, but are not limited to, viruses, bacteria, parasites, protozoa and fungi.

In one embodiment, the present invention relates to a method of detecting one or more antibodies associated with or indicative of SARS-CoV-2 infection, or COVID-19, in a subject. In one embodiment, the method may be performed as a hybridization assay and includes the steps of obtaining a sample from the subject, adding a detector molecule labeled with a detectable moiety directed against a constant region of a human antibody to the sample, applying the sample to an electrode chip coated with a conducting polymer previously embedded or functionalized with one or more capture antigen, and measuring the current in the electrode chip. The detectable moiety may be measured, or the magnitude of the current in the sample may be measured, to determine the presence or absence of a SARS-CoV-2 antibody in the sample. In certain embodiments, hybridization of the SARS-CoV-2 antibody to the capture antigen embedded in the electrode of the sensor results in an increase in current or negative current. For example, in one embodiment, hybridization results in a current in the range of about −10 nA to about −1000 nA.

The present invention provides a method for diagnosing a subject as having or having immunity to COVID-19. In some embodiments, the present invention features methods for identifying subjects who are at risk of spreading SARS-CoV-2 infection or COVID-19, including those subjects who are asymptomatic or only exhibit non-specific indicators of SARS-CoV-2 infection or COVID-19, by detection of the SARS-CoV-2 antibodies as described herein. In some embodiments, the present invention features methods for identifying subjects who are at immune to SARS-CoV-2 infection or COVID-19, by detection of the SARS-CoV-2 antibodies as described herein. In some embodiments, the present invention is also useful for monitoring subjects undergoing treatments and therapies for SARS-CoV-2 infection or COVID-19, and for selecting or modifying therapies and treatments that would be efficacious in subjects having SARS-CoV-2 infection or COVID-19, wherein selection and use of such treatments and therapies promote immunity to SARS-CoV-2, or prevent infection by SARS-CoV-2.

In certain embodiments, the SARS-CoV-2 antibodies detected by way of the system and method of the invention include, but are not limited to, anti-spike protein 1 antibodies, anti-spike protein 2 antibodies, anti-envelope protein antibodies and anti-nucleocapsid antibodies. The present invention may be used to detect an antibody to any SARS-CoV-2 antigen known in the art or discovered in the future.

The invention provides improved diagnosis, therapeutic monitoring, detection of recurrence, and prognosis of SARS-CoV-2 infection or COVID-19. The risk of developing COVID-19 can be assessed by measuring one or more of the SARS-CoV-2 antibodies described herein, and comparing the measured values to reference or index values. Such a comparison can be undertaken with mathematical algorithms or formula. Subjects identified as not having SARS-CoV-2 antibodies can optionally be selected to receive treatment regimens, such as administration of prophylactic or therapeutic vaccines to prevent the onset of SARS-CoV-2 infection or COVID-19.

Identifying a subject before they develop SARS-CoV-2 infection or COVID-19 enables the selection and initiation of various therapeutic interventions or treatment regimens in order to delay, reduce or prevent the spread of SARS-CoV-2 infection or COVID-19. In certain instances, monitoring the levels of at least one SARS-CoV-2 antibody also allows for the course of treatment of SARS-CoV-2 infection or COVID-19 to be monitored. For example, a sample can be provided from a subject undergoing treatment regimens or therapeutic interventions, e.g., drug treatments, vaccination, etc. for SARS-CoV-2 infection or COVID-19. Samples can be obtained from the subject at various time points before, during, or after treatment.

The SARS-CoV-2 antibodies of the present invention can thus be used to generate a risk profile or signature of subjects: (i) who are expected to have immunity to SARS-CoV-2 infection or COVID-19 and/or (ii) who are at risk of developing SARS-CoV-2 infection or COVID-19. The antibody profile of a subject can be compared to a predetermined or reference antibody profile to diagnose or identify subjects at risk for developing SARS-CoV-2 infection or COVID-19, to monitor the progression of disease, as well as the rate of progression of disease, and to monitor the effectiveness of SARS-CoV-2 infection or COVID-19 treatments. Data concerning the antibodies of the present invention can also be combined or correlated with other data or test results for SARS-CoV-2 infection or COVID-19, including but not limited to age, weight, BMI, imaging data, medical history, smoking status and any relevant family history.

The present invention also provides methods for identifying agents for treating SARS-CoV-2 infection or COVID-19 that are appropriate or otherwise customized for a specific subject. In this regard, a test sample from a subject, exposed to a therapeutic agent, drug, or other treatment regimen, can be taken and the level of one or more SARS-CoV-2 antibody can be determined. The level of one or more SARS-CoV-2 antibody can be compared to a sample derived from the subject before and after treatment, or can be compared to samples derived from one or more subjects who have shown improvements in risk factors as a result of such treatment or exposure.

In one embodiment, the invention is a method of diagnosing SARS-CoV-2 infection or COVID-19. In one embodiment, the method includes determining immunity to infection or reinfection by SARS-CoV-2. In some embodiments, these methods may utilize at least one biological sample (such as urine, saliva, blood, serum, plasma, amniotic fluid, or tears), for the detection of one or more SARS-CoV-2 antibody of the invention in the sample. Frequently the sample is a “clinical sample” which is a sample derived from a patient. In one embodiment, the biological sample is a blood sample. In certain embodiments, the biological sample is a serum sample or a plasma sample, derived from a blood sample of the subject.

In one embodiment, the method comprises detecting one or more SARS-CoV-2 antibody in at least one biological sample of the subject. In various embodiments, the level of one or more SARS-CoV-2 antibody of the invention in the biological sample of the subject is compared to a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

In one embodiment, the method comprises detecting one or more SARS-CoV-2 antibody simultaneously in two or more different biological samples of the subject. In one embodiment, the method comprises detecting one or more SARS-CoV-2 antibody simultaneously in a saliva sample of the subject and a blood, plasma or serum sample of the subject.

In one embodiment, the method comprises detecting one or more SARS-CoV-2 antibody sequentially in two or more different biological samples of the subject. In one embodiment, the method comprises detecting one or more SARS-CoV-2 antibody in a saliva sample of the subject prior to or subsequently to detecting one or more SARS-CoV-2 antibody in a blood, plasma or serum sample of the subject.

In one embodiment, the method comprises detecting one or more SARS-CoV-2 antibody in combination with one or more additional biomarker. In one embodiment, one or more additional biomarker is detected concurrently with one or more SARS-CoV-2 antibody. In one embodiment, one or more additional biomarker is detected sequentially either before or after one or more SARS-CoV-2 antibody. The one or more additional biomarker may be any one of a nucleic acid, protein, small molecule, antibody, antibody fragment and the like which are of interest and are present in the sample. In some embodiments, the one or more additional biomarkers are additional disease associated biomarkers. In some embodiments, the one or more additional biomarkers are additional indicators of SARS-CoV-2 infection.

In various embodiments, the subject is a human subject, and may be of any race, sex and age.

Information obtained from the methods of the invention described herein can be used alone, or in combination with other information (e.g., disease status, disease history, vital signs, blood chemistry, etc.) from the subject or from the biological sample obtained from the subject.

The present invention further includes an assay kit containing the electrochemical sensor array and instructions for the set-up, performance, monitoring, and interpretation of the assays of the present invention. Optionally, the kit may include reagents for the detection of at least one SARS-CoV-2 antibody. The kit may also optionally include the sensor reader.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Example 1: SARS-COV-2 (COVID-19) Antibody Test on Saliva and Blood Using EFIRM Technology

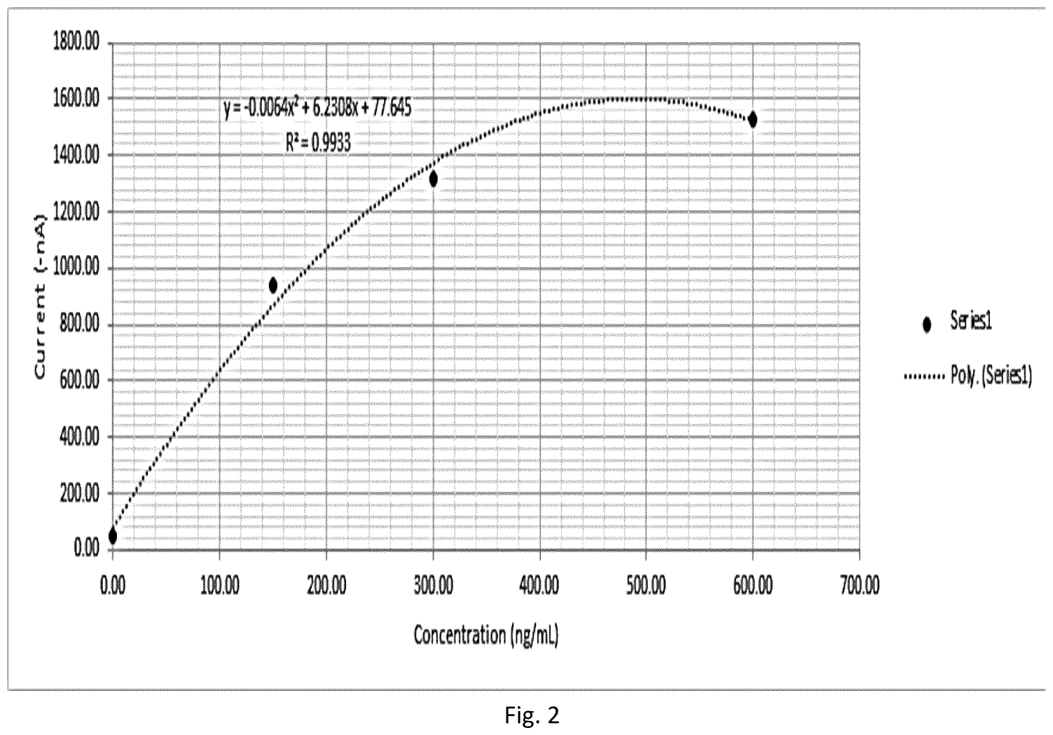

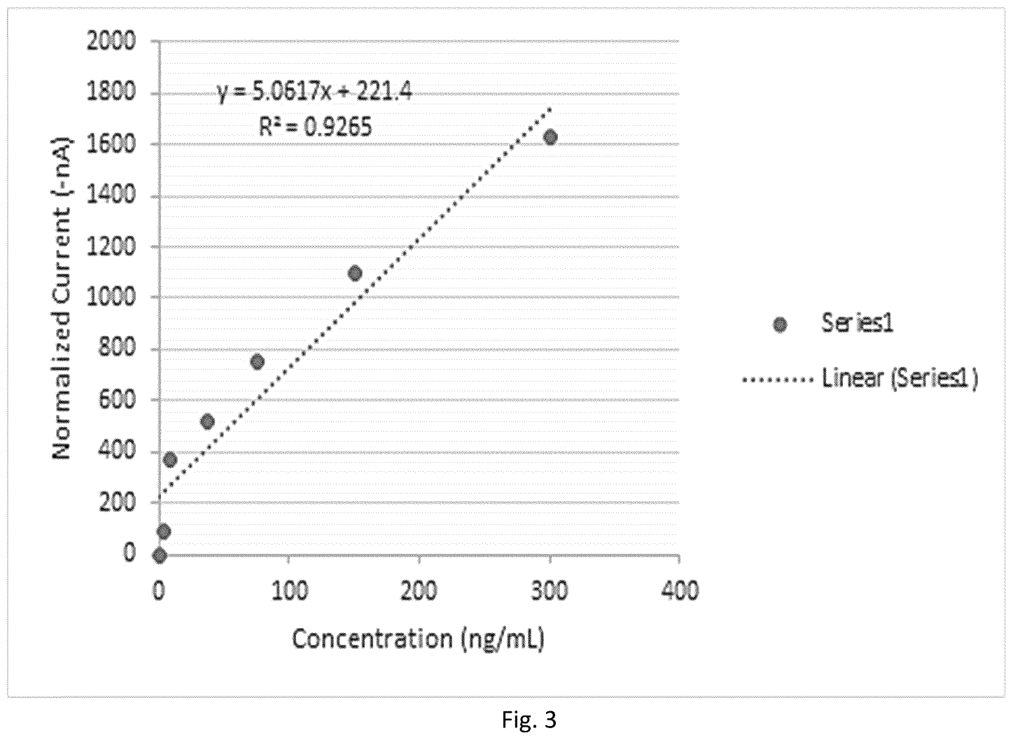

SARS-CoV-2 virus spike proteins 1 and 2 (51 and S2) are the most specific antigens to the virus. S1 has been shown to be the most specific for COVID-19 and S2 is also highly specific. 3 recombinant preparations were obtained from a commercial vendor: S1, a preparation containing a mixture of S1 and S2, and a preparation containing the N protein which is specific for many Coronaviruses. Recombinant human anti-S1 antibody was purchased from a commercial vendor. Using these reagents, an EFIRM assay was designed as shown in FIG. 1. This assay was demonstrated to be linear in the range of 25 ng-300 ng/ml (FIGS. 2-3).

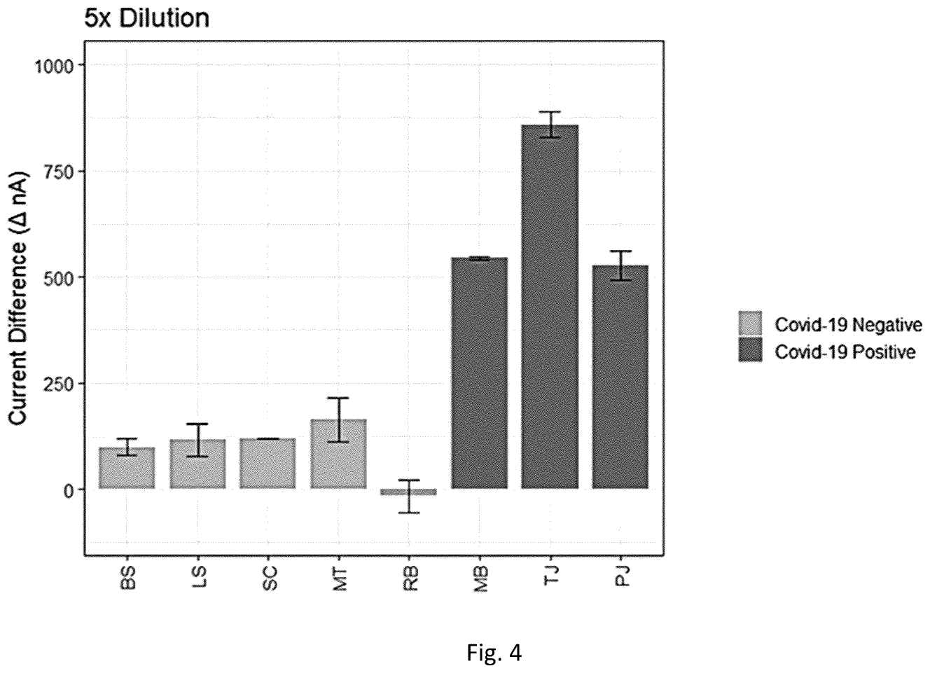

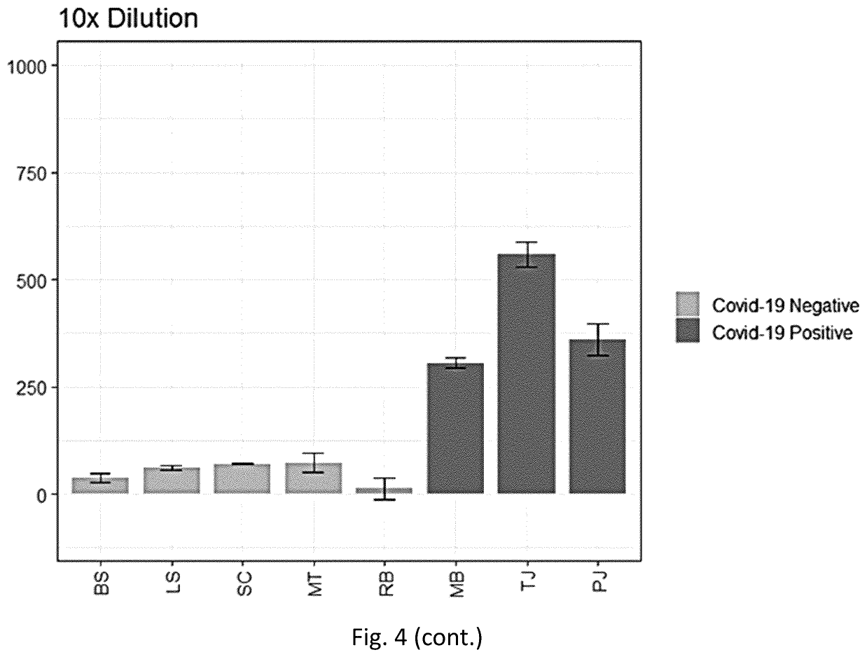

FIG. 4 demonstrates that this assay is capable of detecting the presence of IgG and IgA antibodies to SARS-CoV-2 in 3 convalescent patients with documented COVID-19 infection with symptoms beginning >2 weeks prior to testing. A fourth patient has also been tested and was positive for elevations of antibody.

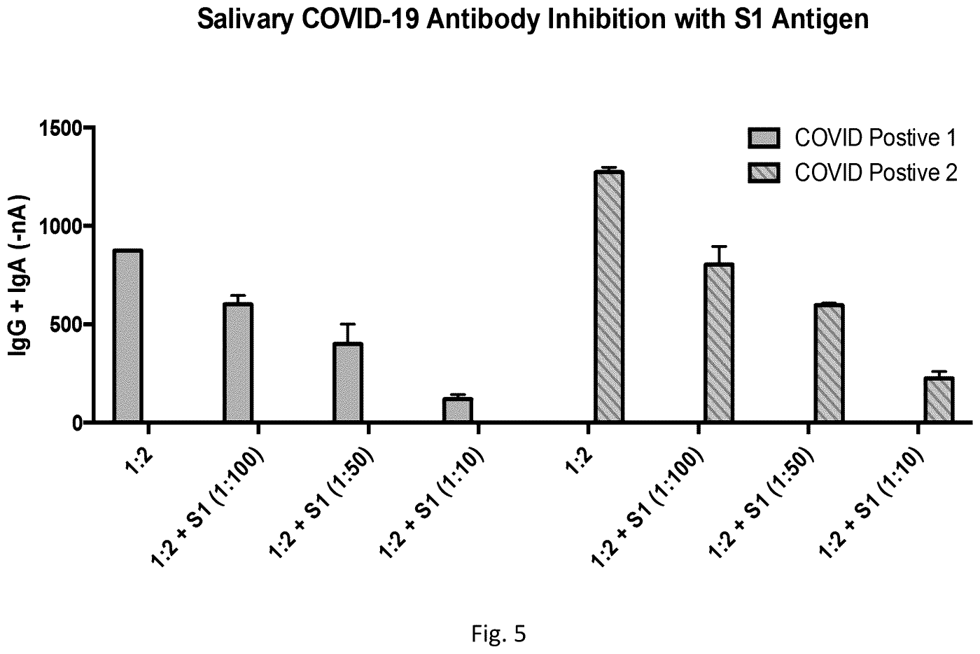

FIG. 5 shows the specificity of the assay, as addition of exogenous S1 antigen at varying concentrations diminished the EFIRM signal in a dose dependent manner.

These data, taken together, demonstrate that an EFIRM assay is capable of detection and quantitation of COVID-19 antibody in biofluids.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A system for detecting a SARS-CoV-2 antibody in a sample, comprising:
a) a multi-well plate comprising an array of sensors, wherein each well comprises an electrode chip including a working electrode, a counter electrode, and a reference electrode; wherein the working electrode of at least one unit is coated with a conducting polymer;
b) at least one SARS-CoV-2 capture antigen, wherein the at least one capture antigen is embedded or functionalized in the conducting polymer;
c) at least one labeled detector molecule, and further wherein the detector molecule is biotin labeled;
d) a multi-well plate washer; and
e) a multi-channel electrochemical reader which controls an electrical field applied onto the array sensors and reports the amperometric current simultaneously.

2. The system of claim 1, wherein at least one capture antigen is selected from the group consisting of a SARS-CoV-2 spike 1 antigen, a SARS-CoV-2 spike 2 antigen, a SARS-CoV-2 envelope antigen, nucleocapsid protein, a protein synthesized from a SARS-CoV-2 open reading frame (ORF), a fragment thereof and a combination thereof.

3. The system of claim 2, wherein the capture antigen comprises a combination of SARS-CoV-2 spike 1 antigen and SARS-CoV-2 spike 2 antigen.

4. The system of claim 1, wherein at least one detector molecule is a secondary antibody specific for binding to an antibody constant region.

5. The system of claim 4, comprising a combination of at least two detector molecules wherein the at least two detector molecules are secondary antibodies specific for binding to an antibody constant region.

6. The system of claim 5, comprising a combination of at least two detector molecules wherein the at least two detector molecules are secondary antibodies specific for binding to an IgG and an IgA constant region.

7. A method of detecting a SARS-CoV-2 antibody in a subject comprising:
obtaining at least one sample of the subject;
mixing a first portion of the at least one sample with a solution comprising a labeled detector molecule;
adding the mixture to a single well of a multi-well plate for use in a system of claim 1, wherein each well of the multi-well plate comprises an electrode chip comprising a working electrode, a counter electrode, and a reference electrode; wherein the working electrode is coated with a conducting polymer embedded with a capture antigen;
applying a cyclic square-wave electric field to the electrode chip; and
measuring the current in the electrode chip, wherein a change in current is correlated to the presence of a SARS-CoV-2 antibody in the sample.

8. The method of claim 7, further comprising at least one washing step, wherein the multi-well plate is washed using an automated plate washer.

9. The method of claim 7, further comprising amplifying the signal, method comprising the steps of:
a) mixing a first portion of the at least one sample with a solution comprising a biotin labeled detector molecule;
b) adding the mixture to a single well of a multi-well plate for use in a system of claim 1, wherein each well of the multi-well plate comprises an electrode chip comprising a working electrode, a counter electrode, and a reference electrode; wherein the working electrode is coated with a conducting polymer embedded with a capture antigen;
c) applying a cyclic square-wave electric field to the electrode chip;
d) adding a first round of streptavidin bound horseradish peroxidase (HRP) to the well;
e) adding a biotin labeled anti-HRP antibody to the well;
f) adding a second round of streptavidin bound HRP to the well; and
g) measuring the current in the electrode chip, wherein a change in current is correlated to the presence of a SARS-CoV-2 antibody in the sample.

10. The method of claim 7, wherein at least one capture antigen is selected from the group consisting of a SARS-CoV-2 spike 1 antigen, a SARS-CoV-2 spike 2 antigen, a SARS-CoV-2 envelope antigen, nucleocapsid protein, any protein synthesized from a SARS-CoV-2 open reading frame (ORF), a fragment thereof and a combination thereof.

11. The method of claim 10, wherein the capture antigen comprises a combination of SARS-CoV-2 spike 1 antigen and SARS-CoV-2 spike 2 antigen.

12. The method of claim 7, wherein at least one detector molecule is a secondary antibody specific for binding to an antibody constant region.

13. The method of claim 12, comprising a combination of at least two detector molecules wherein the at least two detector molecules are secondary antibodies specific for binding to an antibody constant region.

14. The method of claim 13, comprising a combination of at least two detector molecules wherein the at least two detector molecules are secondary antibodies specific for binding to an IgG and an IgA constant region.

15. The method of claim 7, wherein at least one sample is selected from the group consisting of a saliva sample, a blood sample, a plasma sample and a serum sample.

16. The method of claim 7, further comprising diagnosing a subject as having, being at risk of spreading, having been exposed to or having immunity to SARS-CoV-2 infection or COVID-19 when a SARS-CoV-2 antibody is detected in the sample from the subject.

17. The method of claim 16, further comprising administering a therapeutic treatment for COVID-19 to the subject when the SARS-CoV-2 antibody is detected.

18. The method of claim 7, further comprising diagnosing a subject as being at risk of SARS-CoV-2 infection or COVID-19 when a SARS-CoV-2 antibody is not detected in the sample from the subject.

19. The method of claim 18, further comprising administering a prophylactic treatment for COVID-19 to the subject when the SARS-CoV-2 antibody is not detected.
